(19) United States
(12) Patent Application Publication
(10) Pub. No.: US 2026/0165806 A1
(43) Pub. Date: Jun. 18, 2026

(54) STERILE MONITOR DRAPE

(71) Applicant: InSurgery, LLC, Lunenburg, MA (US)

(72) Inventors: Russell Stanton, Lunenburg, MA (US); Edward Daley, Maynard, MA (US)

(21) Appl. No.: 19/346,654

(22) Filed: Oct. 1, 2025

Related U.S. Application Data
(60) Provisional application No. 63/733,579, filed on Dec. 13, 2024.

(51) Int. Cl. A61B 46/10

(52) U.S. Cl. CPC A61B 46/10

(57) ABSTRACT

A monitor sterile drape includes a semi-rigid, form-fitted, conforming monitor cover with a monitor screen cover sheet, walls rearwardly extending from the cover sheet for engaging the monitor, wings extending rearward with respect to the monitor screen cover sheet and biased to engage opposing portions of a monitor housing and to wrap around to a back side of the monitor housing, and at least one fence on the cover sheet. The drape further includes an outer flexible sterile barrier over the monitor cover, but removeable therefrom before use of the monitor. The outer flexible sterile barrier is configured to cover the monitor screen cover sheet and includes wraps configured to engage the monitor screen cover sheet wings.

### RELATED APPLICATIONS

This application claims benefit of and priority to U.S. Provisional Application Ser. No. 63/733,579 filed Dec. 13, 2024, under 35 U.S.C. § § 119, 120, 363, 365, and 37 C.F.R. § 1.55 and § 1.78, which is incorporated herein by this reference.

### FIELD OF THE INVENTION

This subject invention relates to drapes for monitors.

### BACKGROUND OF THE INVENTION

There is a growing trend to bring more integrated technology (imaging, robotics, surgical navigation) into the operating room (OR) to improve surgery. Surgeons use these tools to optimize care and reduce the likelihood of error. Most of these technologies send their outputs to monitors that surgeons can interact with. A good example is the trend to plan screw placement for a spine surgery with images displayed on a monitor taken minutes earlier. It is an advantage to plan such surgeries in real-time.

To interact with the monitor touch screen, the surgeon typically has three options. She can direct a non-sterile staff member to interact with the screen. However, working through another person has its drawbacks. The surgeon could also “break scrub” and interact with the monitor directly. This is the more intuitive approach but it requires the surgeon “scrub in” again. Scrubbing in again can take a few minutes which costs the hospital money plus the cost of the new gloves and gowns and taking the time to do so can break the flow of the team. That flow is often hard to quantify but given that most ORs strictly require that only essential verbal communication be made during the surgical procedure it is evident that a good team does not like anything that distracts their focus.

The last option is to drape the touch screen with a sterile drape that allows the surgeon to interact with the monitor. This option is desirable as long as the drape does not significantly detract from the user experience. See U.S. Pat. Nos. 11,298,201; 5,970,980; 5,957,831; 7,389,872; and CA 2669684 incorporated herein by this reference.

Monitor drapes are also used in clean rooms and in other industries.

### BRIEF SUMMARY OF THE INVENTION

Some of the challenges with common monitor drapes are they tend to be oversized to go on easily and to fit a range of monitors. While this flexibility can be desirable it means that they rarely fit any one monitor very well. Many drapes often have a large plastic bag component that needs to be cinched properly to keep the touch interface from moving as the user interacts with the touch screen. This plastic bag also needs to be guarded for the rest of the case to avoid accidental and hard to detect contamination. Not everyone in the OR is sterile. If there is a big sterile bag attached to the monitor, it may be difficult for a non-sterile staff member to assist in moving the monitor. Also, big sterile bags can block a staff member's line of sight. Some drapes require the use of tape to be fixed in place. Tape can leave behind residue. Some monitor drapes have an adhesive back and stick to the surface of the monitor. These can be a challenge to install and often leave the installer struggling to remove bubbles and realign fit. Once the installer commits to attaching the drape, it is very difficult to take off and reposition the drape without risk of contamination.

In summary, the core problems with some sterile monitor drapes are that they are difficult and time consuming to install and they are large and hard to manage often requiring two hands to control. It is especially hard for a smaller staff member to install a large item without accidentally contaminating it. Some drapes often don't fit well which detracts from the touch screen experience. Some drapes are bulky and need to be monitored throughout the case to prevent contamination. Also, if adhesive is used for fixation, there is a risk of contamination associated with not properly removing adhesive by cleaning.

One proposed solution is designed to be a custom fit for specific monitor sizes. It has two main components that are both preferably sterilized.

The drape includes a monitor cover, preferably, a thermoformed sheet of clear plastic that is sized to fit a specific monitor. This component is what the surgeon will touch to interface with the touch screen. The geometry of this component is slightly oversized to make installation easy. Once it is placed over the monitor, spring loaded “wings” on the back of the cover are released to apply a clamping force to the monitor. This clamping force holds it in place without the need for other fixation (tape, Velcro®, magnets, etc.). The range of Young's modulus (modulus of elasticity) for the monitor cover may range from 1.8-4.5 Gigapascals (GPa) with a thickness range from 0.005″ to 0.035″.

Another component of the drape is a flexible sterile barrier is preferably a thin sheet (plastic or paper, etc.) that is used to wrap the outward facing surfaces of the monitor cover. It is held in place with respect to the monitor cover with a light tack adhesive or other suitable means (folding, thermally welded, static cling, etc.). Having this protective layer offers two main advantages. First, it is meant to protect the monitor cover from accidental contamination prior to the surgeon's interaction with it. Monitors are quite large and are an easy target for accidental contamination. Even minor air turbulence of a person walking by quickly has the potential to stir up hard to detect particulate. Also, when items are sterile it is recommended that the staff do not turn their back to them. With a protective cover in place, the staff can freely move about knowing the surface they ultimately need to be sterile is protected. Essentially this added barrier reduces cognitive load on the staff. A second advantage is that if the staff chooses, they can opt to have a non-sterile staff member install the monitor cover. Because they will have no direct contact with the sterile monitor cover the surgeon touches, the risk of contamination is low. Whenever non-sterile staff can assist, it offers flexibility that can save the hospital time and money.

Featured is a monitor sterile drape comprising a semi-rigid, form-fitted, conforming monitor cover with a monitor screen cover sheet and wings extending rearward with respect to the monitor screen cover sheet and biased to engage opposing portions of a monitor housing and to wrap around to a back side of the monitor housing. An outer flexible sterile barrier resides over the monitor cover and is removeable therefrom before use of the monitor.

In one preferred embodiment, the monitor cover further includes at least one wall extending rearward for the monitor screen cover sheet for engaging the monitor housing. There can be four walls extending rearward from the monitor screen coversheet forming a box-like monitor cover. The monitor sterile drape may further include an upwardly extending fence on the monitor cover preventing grasping of the top of the monitor and a downwardly extending fence on the monitor cover preventing grasping of the bottom of the monitor. Preferably, the monitor screen cover sheet wings extend from right and left sides of the monitor cover sheet.

The outer sterile barrier may include wraps over and underlying the wings of the monitor cover sheet.

The monitor screen cover sheet wings may include means for controlling the wings during drape installation such as a longitudinally extending member on each wing.

Also featured is a method of draping a monitor used in a procedure. Before the procedure, opposing wings of a monitor cover fitted with an outer sterile barrier are spread apart and the monitor cover and sterile barrier are installed on the monitor. The opposing wings are released to wrap around to a backside of the monitor. Before the monitor is used, the outer sterile barrier is removed from the monitor cover.

In one example, the monitor cover is semi-rigid, form-fitted, and conforms to the monitor. The monitor cover can include a monitor screen cover sheet and at least one wall extending rearward from the monitor screen cover sheet. Preferably, the outer sterile barrier is flexible.

Removing the outer sterile barrier may include ripping the outer sterile barrier from the monitor cover. The method may further include repositioning the monitor after the monitor cover and outer sterile barrier are installed by touching one or more of the opposing wings before the outer sterile barrier is removed from the monitor cover.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 shows an example of a typical monitor in an operating room environment;

FIG. 2 is a schematic view of a prior art drape for an operating room monitor;

FIG. 3 is an exploded view of a new monitor sterile drape in accordance with an example;

FIG. 4 is a schematic rear view of an example of a monitor cover;

FIG. 5 is a schematic view showing the monitor cover of FIG. 4 installed on a monitor;

FIG. 6 is a schematic view showing the rear side of the monitor cover clamped to the monitor housing;

FIG. 7 is a schematic front view showing the flexible sterile barrier over the monitor cover;

FIG. 8 is a schematic view showing removal of the flexible sterile barrier from the monitor cover;

FIGS. 9-10 are schematic views showing how the monitor can be repositioned in a sterile fashion; and

FIG. 11 is a schematic view of another example of a monitor cover.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

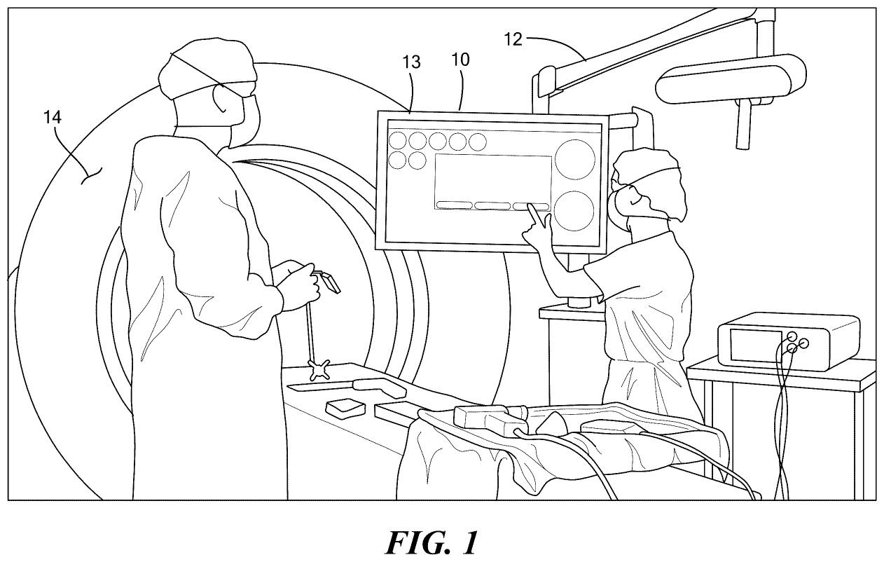

Monitor 10, FIG. 1 (e.g., a touch screen 13 type monitor) is shown in an operating room mounted to swing arm 12 for displaying images from imager 14, for controlling various equipment settings, and the like.

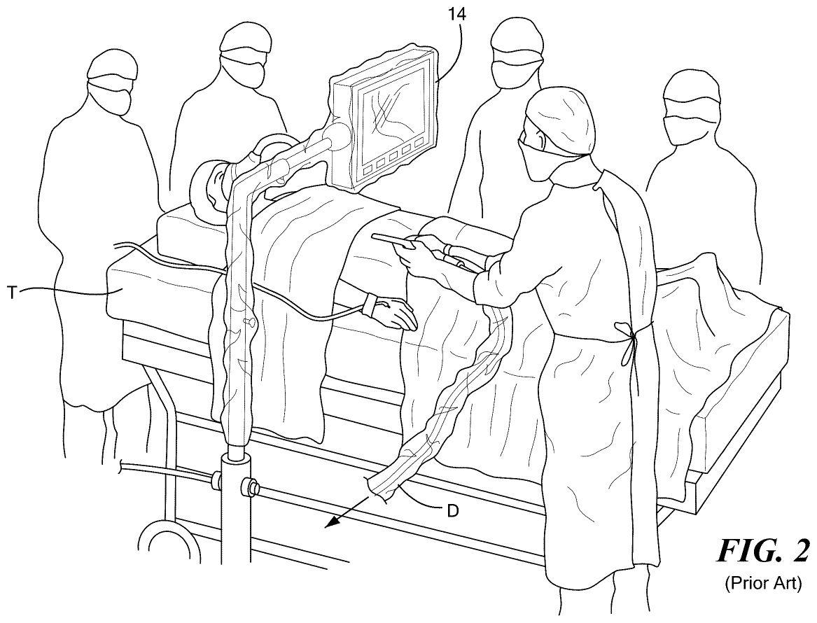

Monitor drape 14, FIG. 2 in accordance with the prior art is a flexible, large plastic bag type construction which may not fit well, can be difficult to install, hard to manage, and subject to contamination and which presents other challenges as discussed above. See U.S. Pat. No. 5,970,980.

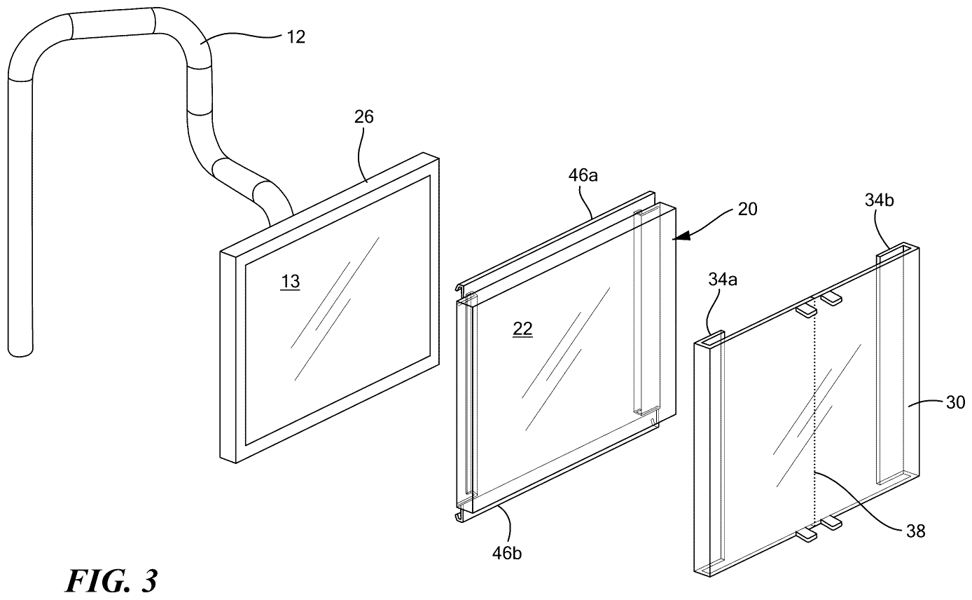

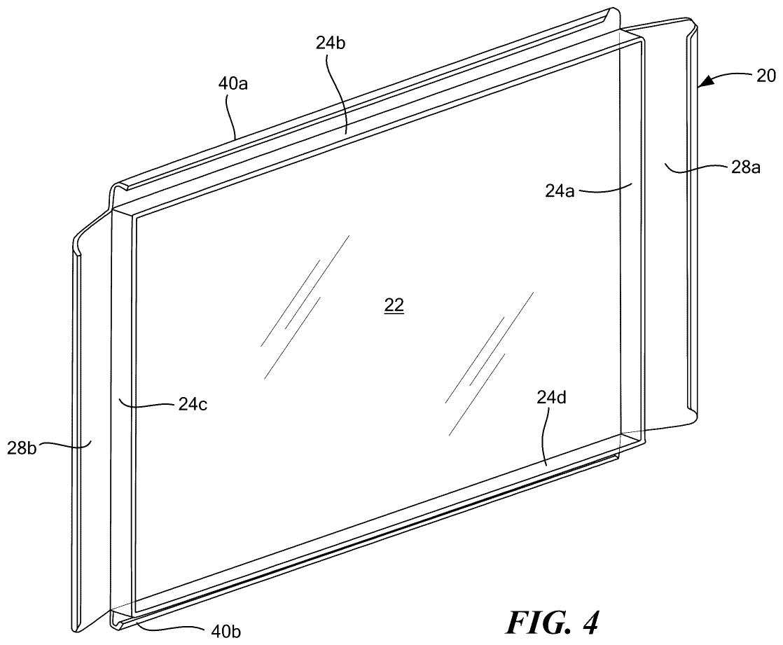

One preferred monitor sterile drape includes a semi-rigid, form-fitted, conforming box-like monitor cover 20, FIG. 3 including monitor screen cover sheet 22 and four side walls 24a, 24b, 24c, and 24d, FIG. 4 extending rearward from cover sheet 22 and designed to form fit on a monitor and, in one example, to engage the top, bottom, and both sides of the monitor housing 26, FIG. 3. The semi-rigid clear plastic material of the monitor cover can be 0.005″-0.035″ inches thick and made of thermoformed PET, PETG, Polycarbonate, Polyethylene, PVC, Polypropylene, Acrylic. Or other clear plastic such as PETE, PP, Polystyrene, APET, PPE or Butyrate.

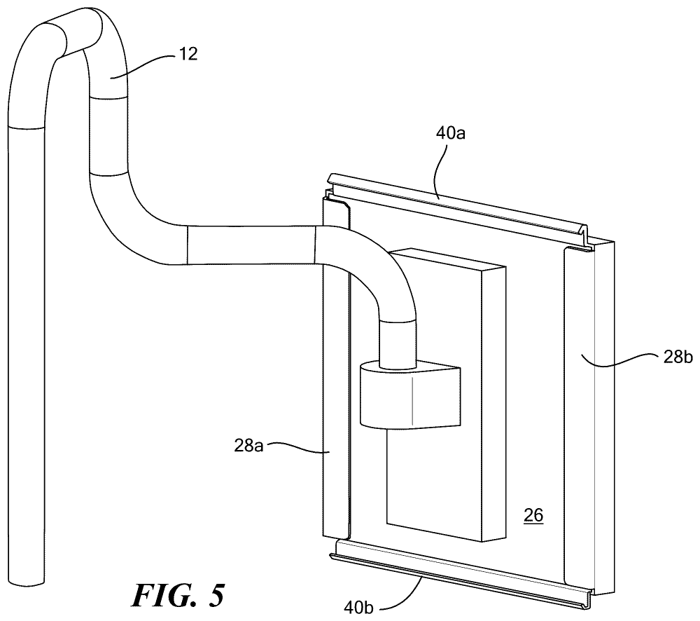

In this example, right and left side wings 28a, 28b, FIG. 4 extend rearward from cover sheet 22 and are flexible but biased inward to engage the right and left sides of the monitor housing and biased to wrap around the back side of the monitor housing as shown in FIG. 5. Monitor cover 20 may also include upwardly and outwardly extending fence 40a and downwardly and outwardly extending fence 40b preventing grasping of the top and bottom of the monitor which could possibly contaminate the operating room environment due to the unsterile nature of the back of the monitor housing.

Returning to FIG. 3, the preferred monitor drape also includes flexible sterile barrier 30 over the monitor cover and removable therefrom before surgery/imaging and use of the monitor. The flexible sterile barrier may be plastic material 0.001″-0.015″ inches thick. Polyethylene (or similar) may be used. Alternatively, a nonwoven sterile fabric, or paper sheet may also be used in lieu of plastic.

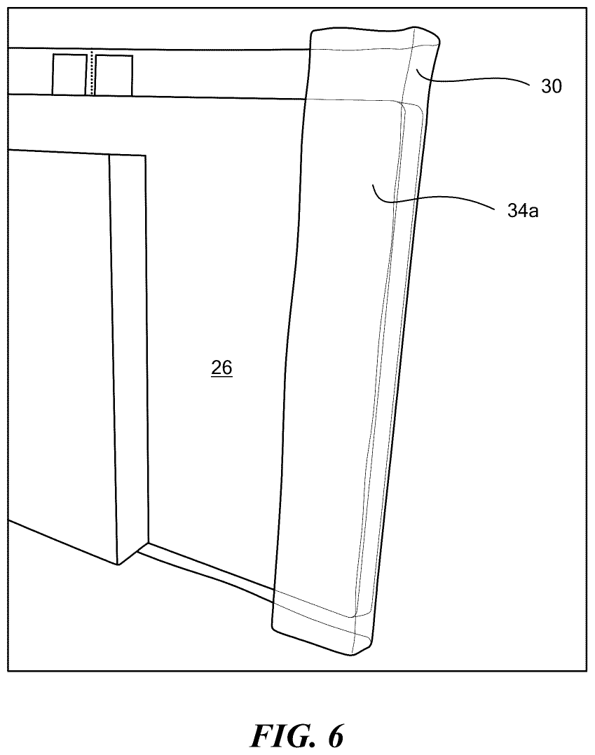

Barrier 30 can also include sheet 32 over monitor cover sheet 22 and side wraps 34a, 34b designed to lie against the backside of the monitor housing around and then underneath the side wings 28a, 28b, FIG. 4 of the monitor cover as shown in FIG. 6. The barrier side wraps thus engage the monitor cover sheet 22 side wings 28a, 28b.

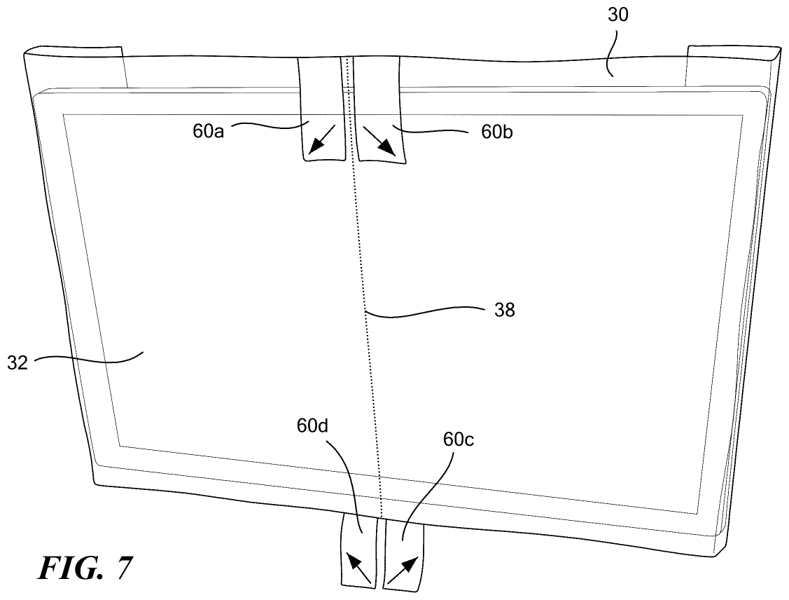

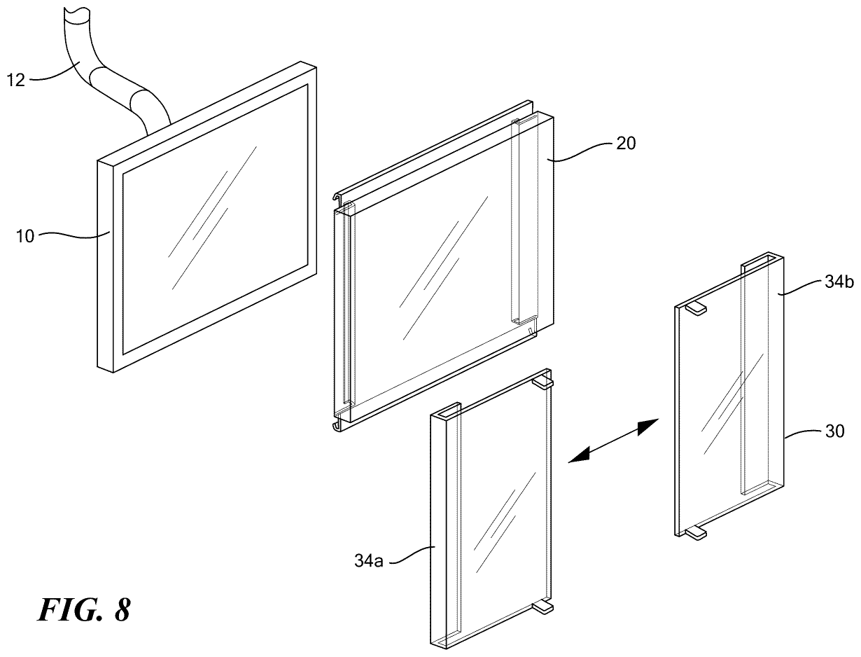

Barrier 30 is preferably designed to tear away from the monitor cover, for example, by top to bottom middle perforations 38, FIG. 7 and sterile tabs 60a-60d (two at the top and two at the bottom on opposite sides of perforations 38). See also FIG. 8.

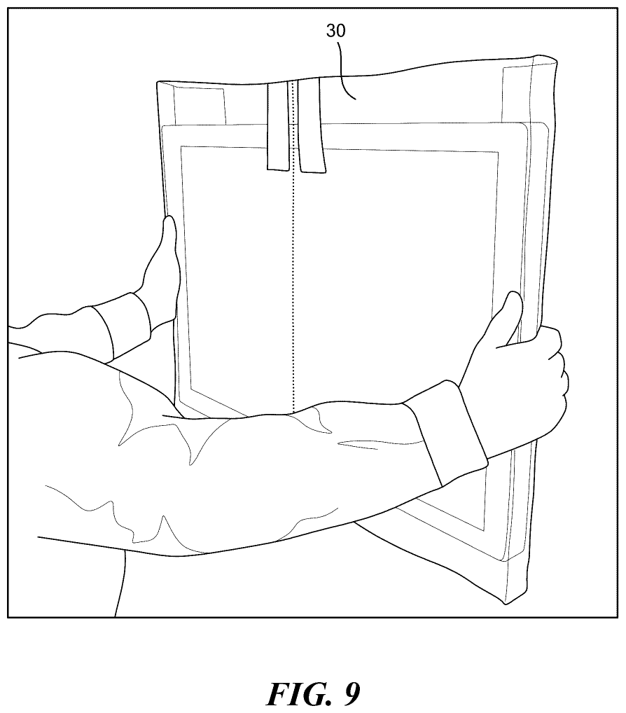

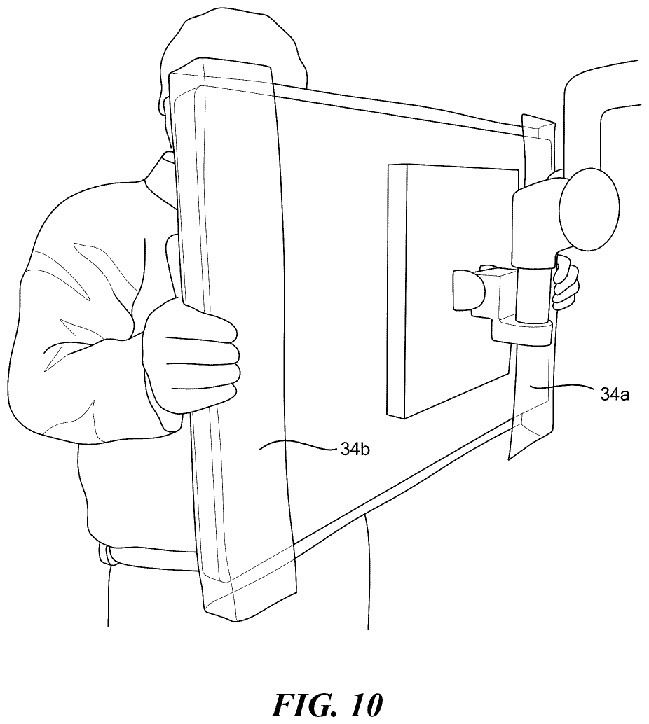

As shown in FIGS. 9-10, the monitor can be safely repositioned if needed by grasping the right and left sides where the side wraps of the sterile barrier are touched but not the wings of the monitor cover or the back of the monitor housing.

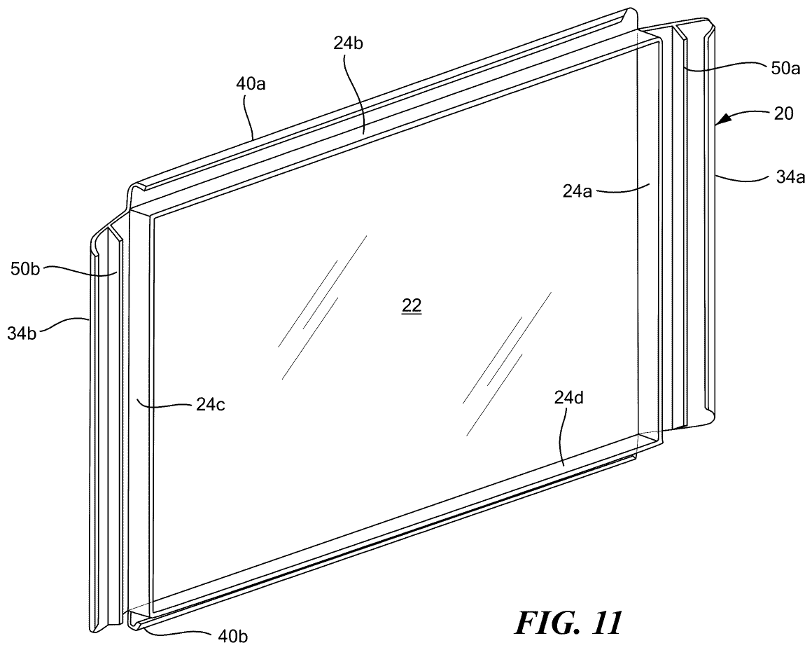

In one embodiment, monitor cover 20, FIG. 11 wings 34a, 34b include a longitudinally extending member such as a hem or boss 50a, 50b allowing the installer to control the spring force release of the side wings 34a, 34b during installation on a monitor to reduce air turbulence and to allow installers with smaller hands to more aptly control the wings. Other wing control means are possible.

The rigidity of the box shape of the thermal formed cover makes its large size more rigid and thus easier to control with confidence than conventional monitor drapes. Its box-like geometry also provides a means of alignment and support. The spring-loaded wings have a very low learning curve for proper use. The clamping force provided by the wings eliminates the need for secondary means of fixation such as tape, magnets, Velcro®, or the like. This clamping force also holds the shape of the thin plastic monitor screen cover sheet against the monitor which improves touch screen interactions. The protective cover also allows for greater protection and more flexibility at the time of installation. The fences and clearly designated hand holds (wings) allow the user to orient the monitor with confidence and reduce the risk of contamination.

In use, the user bends open the monitor cover with its protective barrier in place so that the monitor cover wings are spread apart so they can fit past the sides of the monitor. Next, the user places the monitor cover and sterile barrier onto the monitor. Once this is done, the weight of the drape is supported by the monitor due to its geometry. Next, the user releases the wings of the monitor cover and they provide a clamping force on the backside of the monitor. Then, at the appropriate time, the tear-away barrier is removed.

The monitor cover also has features to help prevent a sterile user from accidentally touching the non-sterile back surface of the monitor. This might occur when the user attempts to reorient (tilt) the monitor for a better view. The fences 40a, 40b on the top and bottom side of the cover to prevent the user's fingers from accidentally wrapping around to the back of the monitor from the top or bottom.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. A monitor sterile drape comprising:
a semi-rigid, form-fitted, conforming monitor cover including:
a monitor screen cover sheet, and
wings extending rearward with respect to the monitor screen cover sheet and biased to engage opposing portions of a monitor housing and to wrap around to a back side of the monitor housing; and
an outer flexible sterile barrier over the monitor cover and removeable therefrom before use of the monitor.

2. The monitor cover of claim 1 further including at least one wall extending rearward for the monitor screen cover sheet for engaging the monitor housing.

3. The monitor of claim 2 in which there are four walls extending rearward from the monitor screen coversheet forming a box-like monitor cover.

4. The monitor sterile drape of claim 1 further including an upwardly extending fence on the monitor cover preventing grasping of the top of the monitor.

5. The monitor sterile drape of claim 1 further including a downwardly extending fence on the monitor cover preventing grasping of the bottom of the monitor.

6. The monitor sterile drape of claim 1 in which the monitor screen cover sheet wings extend from right and left sides of the monitor cover sheet.

7. The monitor sterile drape of claim 1 in which the outer sterile barrier includes wraps over and underlying the wings of the monitor cover sheet.

8. The monitor sterile drape of claim 1 in which the monitor screen cover sheet wings include means for controlling the wings during drape installation.

9. The monitor sterile drape of claim 8 in which the means for controlling include a longitudinally extending member on each wing.

10. A method of draping a monitor used in a procedure, the method comprising:
before the procedure, spreading opposing wings of a monitor cover fitted with an outer sterile barrier;
installing the monitor cover and sterile barrier on the monitor;
releasing the opposing wings to wrap around to a backside of the monitor; and
before use of the monitor, removing the outer sterile barrier from the monitor cover.

11. The method of claim 10 in which the monitor cover is semi-rigid, form-fitted, and conforms to the monitor.

12. The method of claim 11 in which the monitor cover includes a monitor screen cover sheet and at least one wall extending rearward from the monitor screen cover sheet.

13. The method of claim 10 in which the outer sterile barrier is flexible.

14. The method of claim 13 in which removing the outer sterile barrier includes ripping the outer sterile barrier from the monitor cover.

15. The method of claim 10 further including repositioning the monitor after the monitor cover and outer sterile barrier are installed by touching one or more of the opposing wings before the outer sterile barrier is removed from the monitor cover.

16. The method of claim 15 in which the outer sterile barrier includes wrap portions lying over and under the opposing wings of the monitor cover.

17. A monitor sterile drape comprising:
a semi-rigid, form-fitted, conforming monitor cover including:
a monitor screen cover sheet,
walls rearwardly extending from the cover sheet for engaging the monitor,
wings extending rearward with respect to the monitor screen cover sheet and biased to engage opposing portions of a monitor housing and to wrap around to a back side of the monitor housing, and
at least one fence on the cover sheet; and
an outer flexible sterile barrier over the monitor cover and removeable therefrom before use of the monitor, the outer flexible sterile barrier configured to cover the monitor screen cover sheet and including wraps configured to engage the monitor screen cover sheet wings.

18. The monitor sterile drape of claim 17 including an upwardly extending fence on the monitor cover preventing grasping of the top of the monitor.

19. The monitor sterile drape of claim 17 including a downwardly extending fence on the monitor cover preventing grasping of the bottom of the monitor.

20. The monitor sterile drape of claim 17 in which the monitor screen cover sheet wings extend from right and left sides of the monitor cover sheet.

21. The monitor sterile drape of claim 17 in which the monitor screen cover sheet wings include means for controlling the wings during drape installation.

22. The monitor sterile drape of claim 21 in which the means for controlling include a longitudinally extending hem or boss on each wing.
